(19) United States
(12) Patent Application Publication
(10) Pub. No.: US 2021/0401281 A1
(43) Pub. Date: Dec. 30, 2021

(54) ARTICULATED DENTAL PHOTOGRAPHY KIT

(71) Applicant: DENTAL MONITORING, PARIS (FR)

(72) Inventors: Philippe SALAH, PARIS (FR); THOMAS PELLISSARD, PARIS (FR); Cedric LANCON, VILLERS FARLAY (FR); Laurent DEBRAUX, PARIS (FR)

(21) Appl. No.: 17/289,845

(22) PCT Filed: Oct. 29, 2019
(86) PCT No.: PCT/EP2019/079565

§ 371 Date: Apr. 29, 2021

(30) Foreign Application Priority Data
Oct. 30, 2018 (FR) 1860037

(51) Int. Cl. A61B 1/24; A61B 1/00; A61B 1/32; A61B 5/00

(52) U.S. Cl. CPC A61B 1/24; A61B 5/6898; A61B 1/32; A61B 1/00147

(57) ABSTRACT

A dental photography kit that includes a support, taking the form of a housing. The support includes a support attachment part. The kit includes a dental retractor that defines a retractor opening and that includes a retractor attachment part that is removably attached to the support attachment part when the retractor is in the mounted position on the support. The kit includes a camera that is removably attached to the support in an operating position in which the camera is oriented to receive an at least partial image of the retractor opening. The retractor attachment part is articulated on the support attachment part.

The present invention relates to a dental photography device, in particular for implementing a method as described in the international application PCT/EP2015/074896.

### TECHNICAL FIELD

### State of the Art

PCT/EP2015/074897 describes an acquisition kit that allows photos to be taken of the teeth of a patient. This acquisition kit comprises:
a dental retractor intended to be placed in the mouth of the patient;
a cellphone provided with a camera.

The acquisition, by the patient, of photos representing certain regions of the mouth, and in particular the palate, does however remain difficult. The search for photo acquisition conditions is also complex.

US 2018/0228359 describes a dental image acquisition device comprising a dental retractor and a cellphone.

One objective of the present invention is to respond at least partially to the these problems.

### SUMMARY OF THE INVENTION

The invention proposes a dental photography kit, the kit comprising:
a support comprising a support attachment part;
a dental retractor defining a retractor opening of axis X and comprising a retractor attachment part removably fixed to the support attachment part when the retractor is in mounted position on the support; and
a camera, preferably incorporated in a cellphone, that is rigidly or removably attached to the support in an operating position in which the camera is oriented to receive an at least partial image of the retractor opening.

According to a first main aspect of the invention, the retractor attachment part is articulated on the support attachment part.

As will emerge more clearly hereinafter in the description, the support advantageously makes it possible to position the camera with respect to the dental retractor, which makes it possible to speed up the setting of the camera when taking photos, while the articulation of the retractor attachment part with respect to the support attachment part, and preferably the articulation of the retractor with respect to the support, makes it possible to easily modify the regions of the mouth observed by the cameral through the retractor opening.

The acquisition of the photos by the patient is thereby facilitated.

An articulation is a link allowing a rotation about at least one axis of rotation.

In a preferred embodiment, the retractor attachment part is pivotably mounted on the support attachment part. The articulation is therefore a pivot link, preferably pivoting about a pivoting axis, preferably horizontal in the operating position. It then allows only a rotation about this axis. Preferably, the pivoting allows, at least in extreme lower and upper pivoting positions, photos to be taken of the palate and of the tongue, respectively.

The pivoting axis can be fixed with respect to the support. Preferably, the pivoting axis is movable in translation with respect to the support, in a direction that is different from that of the pivoting axis, preferably in a direction at right angles to the pivoting axis, as in accordance with the second main aspect of the invention described hereinbelow.

The support attachment part can be mounted to rotate, about the pivoting axis, on a body of the support. The retractor attachment part can be mounted to rotate, about the pivoting axis, on the support attachment part.

In the preferred embodiment of the invention, in the mounted position of the retractor, the retractor attachment part is magnetically attached to the support attachment part.

Preferably, the retractor is attached, removably, preferably magnetically, by precisely two retractor attachment parts cooperating with two respective support attachment parts. Preferably, each support attachment part being mounted to rotate about a respective pivot, the two pivots preferably being of axis Ya and Yb.

Preferably, the two retractor attachment parts are magnets, preferably embedded in the mass of the retractor.

Preferably, each support attachment is a magnet oriented to attract a metal insert or a magnet of a corresponding retractor attachment. Preferably, the retractor attachment part is a metal insert and the support attachment part is a magnet. Advantageously, tests have shown that the retractor attachment part is thus better resistant to stoving.

According to a second main aspect of the invention, when the retractor is in the mounted position on the support, at least one retractor attachment part, preferably each retractor attachment part, is movable in translation, along a respective axis of translation, with respect to the support.

In other words, the retractor attachment part can be separated from the support on the axis of translation.

As will emerge more clearly hereinafter in the description, said translation advantageously facilitates the taking of photos or of films of dental arch parts which are not within the longitudinal axis of the support.

Preferably, the axis of translation is substantially parallel to the axis X of the retractor opening and/or substantially at right angles to a pivoting axis of the retractor attachment part with respect to a support attachment part to which said retractor attachment part is attached.

Preferably, two planes at right angles to the translation axis and to the axis of the retractor opening X, respectively always form between them an angle of less than 30°, preferably less than 20°, and/or preferably greater than 5°, preferably greater than 10°.

Preferably, two planes at right angles to the translation axis and to the pivoting axis of a retractor attachment part, respectively, always form between them an angle greater than 80° and less than 110°, preferably an angle of 90°.

Preferably, when, in the mounted position of the retractor, the position and/or the geometry of the retractor changes, the translation axis of a retractor attachment part always extends in one and the same plane. Preferably, this plane forms, with the median longitudinal plane of the support, an angle of less than 30°, preferably less than 20°, and/or preferably greater than 5°, preferably greater than 10°.

Preferably, the amplitude of the translation is limited. Preferably, the maximum amplitude is less than 2 cm, preferably less than 1 cm and/or greater than 0.5 cm.

In one embodiment, at least one of the support attachment and retractor attachment parts, preferably the support attachment part, is telescopic on the translation axis.

According to a third main aspect of the invention, the camera is incorporated in a cellphone and the support comprises
a body, preferably in the form of a housing, and
a door mounted to pivot on the body about a door pivot of axis P, preferably substantially horizontal in the operating position, between a closed position in which it holds the cellphone clamped against a receiving face of the body, and an open position in which it allows the cellphone to be extracted,

the door pivot being mounted to slide with respect to the body, for example in a rail of the body, along a sliding axis Z which is not parallel to the axis P, preferably on a sliding axis that is coplanar to the axis P and preferably substantially at right angles to the axis P.

Preferably, two planes at right angles to the axes P and Z, respectively, always form between them an angle greater than 80° and less than 110°, preferably an angle of 90°.

Preferably, the sliding axis Z forms, with the general plane of the receiving face of the body, an angle greater than 80° and less than 110°, preferably an angle of 90°.

As will emerge more clearly hereinafter in the description, the door can thus be separated from the receiving face, while remaining, in the closed position, substantially parallel to the receiving face. The support can thus advantageously be adapted to cellphones having different thicknesses.

Preferably, the support comprises a return member, preferably a spring or a magnet, disposed so as to oppose, preferably elastically, a separation of the door pivot away from the plane of the receiving face, in particular when the door is held parallel to the receiving face. The action of the return member advantageously contributes to the attachment of the cellphone.

Preferably, in the open position of the door, the cellphone rests on a receiving face of the support, preferably flat, and the support comprises only first and second abutments,
the first abutment, preferably a lateral abutment, limiting the slipping of the cellphone on the receiving face in a first direction and in just one direction, when the door is in the open position, and
the second abutment, preferably a bottom abutment, limiting the slipping of the cellphone on the receiving face, in a second direction and in just one direction, when the door is in the open position.

Advantageously, the support is thus suitable for receiving cellphones of different lengths and/or widths.

Preferably, the first and second directions, that are different form an angle preferably greater than 60° and/or less than 120°, preferably of approximately 90°.

Preferably, the support takes the form of a housing emerging outward substantially only through a proximal opening, blocked by the cellphone in the closed position, and a distal opening, emerging in the retractor opening.

According to a fourth main aspect of the invention, the camera, of optical axis O, is incorporated in a cellphone and the cellphone is mounted to be movable on the support in a plane at right angles to said optical axis.

As will emerge more clearly hereinafter in the description, the mobility of the cellphone allows a very accurate centering of the lens on the longitudinal axis of the support.

Preferably, the support comprises a housing and a frame for attaching the cellphone, the frame being guided in translation on the housing.

Preferably, the frame comprises marks identifying the position of the frame with respect to the housing and the housing comprises a marker facing which each identification mark can be positioned. Preferably, the identification marks correspond to cellphone models. In other words, to correctly position a cellphone on the support, the identification mark corresponding to this cellphone simply has to be positioned facing the marker.

Likewise, the marker can be on the frame and the identification marks on the housing.

Preferably, the mobility of the cellphone is indexed, that is to say is done by jumps between predetermined stable positions. Conventionally, an elastic ratchet of the frame can for example cooperate with corresponding holes on the housing.

In one embodiment, the housing comprises a means, that can be selectively activated and deactivated by the operator, for locking the cellphone in position.

Preferably, the mobility of the cellphone is limited to a translation along an axis or to two translations along two axes, preferably substantially at right angles to one another.

Obviously, the features, whether optional or not, of the various main aspects of the invention can be combined.

In particular, at least one, preferably each, attachment of the retractor on the support allows a rotation, preferably a pivoting about the pivoting axis, and a separation, along the axis of translation, of the attachment part the retractor with respect to the support. A kit according to the invention preferably has one or more of the following optional features:
the support bears a mirror that is attached and oriented so that the camera of the cellphone receives a composed image comprising a direct image of the retractor and an image of the retractor opening reflected by the mirror;
the support bears a colorimetric pattern and/or a translucency pattern;
when the camera is incorporated in the support, it comprises wireless communication means, preferably telephony communication means, which facilitates the implementation of the methods described in PCT/EP2015/074897.

### Definitions

“Patient” is understood to mean any person for whom a kit according to the invention can be implemented, whether this person is sick or not, or whether this person is undergoing a treatment or not. A kit according to the invention can be used for an animal other than a human being.

The term “cellphone” conventionally describes an apparatus of iPhone® type. Such an apparatus weighs typically less than 500 g, is provided with a camera allowing it to take films or photos, is capable of exchanging data with another apparatus more than 500 km away from the cellphone, and is capable of displaying said data, and in particular said photos. A “film” is considered to be a set of photos.

“Attach” two parts means to establish a physical link between these two parts, such that any displacement of one of these parts ends causing that of the other part. A part that is mounted in translation, with rotation or with ball joint on another part is attached to that other part. An attachment can be “permanent” or “removable”, depending on whether or not the two parts are adapted for separation, preferably by hand, by an operator. An attachment can be rigid or not, depending on whether or not it allows a relative displacement of the two parts attached to one another.

A magnetic attachment uses the attraction of a magnet and of a metal part, or the attraction between two magnets.

Unless indicated otherwise, the qualifiers used to define positions in space such as “horizontal”, “vertical”, “bottom”, “top”, “right” or “left” are defined, for the purposes of clarity, with reference to an operating position observed by a patient, having the head straight and having arranged his or her lips on the retractor of the kit in its mounted position, as represented in FIG. 7, in which V represents the vertical direction oriented upward.

In the operating position, the median longitudinal plane P₁₂ is the vertical plane which passes through the longitudinal axis of the support. The longitudinal axis L of the support is the axis which extends substantially horizontally in the operating position and which passes through the center of the chamber defined by the support and extending between the retractor and the cellphone.

“Comprise”, “include” and “have” should be interpreted in the broad and nonlimiting sense, unless indicated otherwise.

### BRIEF DESCRIPTION OF THE FIGURES

Other features and advantages of the invention will become more apparent on reading the following detailed description and studying the attached drawing in which:

FIGS. 1A-1B represent, in perspective and seen from the side, the support of a kit according to the invention, with the door in the open position;

FIGS. 2A-2B represent, in perspective, the support represented in FIG. 1 and the cellphone of a kit according to the invention, when the door is in the open position, the cellphone being positioned so as to be able to take photos of the distal opening of the housing;

FIGS. 3A-3B represent, in perspective and in longitudinal cross-section, the support and the cellphone represented in FIG. 2, with the door in the closed position, the locking hook being in unlocked position;

FIGS. 4A-4D represent, in perspective and seen from the side (FIG. 4C), the support and the cellphone represented in FIG. 3, with the door in the closed position, the locking hook being in locked position;

FIGS. 5A-5B represent, seen from the side, a kit according to the invention, in the operating position, in the extreme pivoting positions of the support with respect to the retractor (low and high positions, respectively), only a half of the retractor being represented;

FIG. 6 represents, in perspective, a preferred example of retractor of a kit according to the invention;

FIG. 7 illustrates the use of a kit according to the invention by a patient;

FIG. 8 is an example of photo taken when the support comprises mirrors;

FIGS. 9A-9C illustrate the attachment of a cellphone onto a support, in another preferred embodiment of the invention;

FIGS. 10A-10F illustrate a vertical mobility of the cellphone on the support;

FIGS. 11A-11C illustrate the translation of the retractor attachment parts with respect to the support.

In the various figures, the members that are identical or similar are designated with identical references.

### DETAILED DESCRIPTION

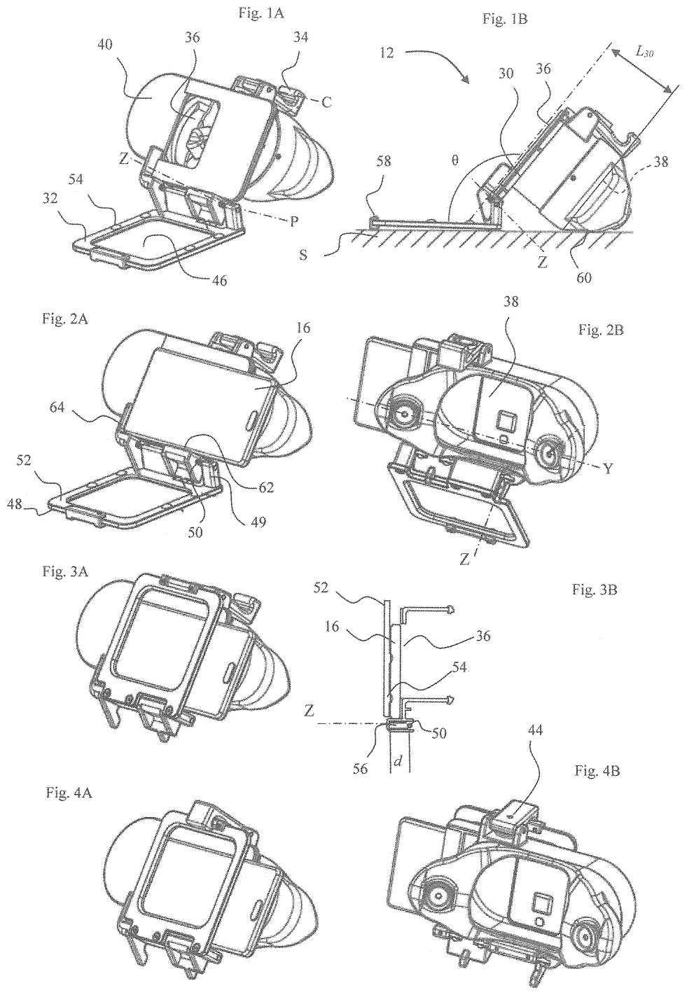

The photography kit 10 represented in FIG. 1 comprises a support 12, a dental retractor 14, and a cellphone 16.

### Retractor

The retractor 14 can have conventional retractor features.

As represented in FIG. 6, it preferably comprises a rim, preferably in two parts 22a and 22b, extending around a retractor opening 24 of axis X and arranged so that the lips of the patient can rest thereon while leaving the teeth of the patient visible through said retractor opening. The lips of the patient are then pulled up around the teeth of the patient.

The two parts of the rim preferably extend substantially symmetrically with respect to a median plane passing through the axis X, preferably vertical, and are preferably linked to one another by a handle 25. The handle 25 facilitates the handling, but also allows the two parts of the rim to be brought closer to one another elastically. The positioning of the lips on the rim is facilitated thereby.

Preferably, the retractor comprises cheek separating lugs 26a and 26b in order for the camera to be able to acquire, through the retractor opening, photos of vestibular faces of the teeth disposed at the bottom of the mouth, such as molars. This feature is particularly advantageous for the implementation of the methods described in PCT/EP2015/074897. In the embodiment represented, the retractor 14 also comprises left 28a and right 28b tabs, substantially at right angles to the axis X, facilitating the handling thereof.

The retractor 14 is preferably made of a biocompatible material, for example made of plastic material.

The retractor is removably mounted on the support, that is to say it can be disposed in mounted and unmounted positions in which it is attached to the support or detached from the support, respectively.

The retractor 14 can be attached to the support by one or more attachments 27a-27a′ and 27b and 27b′, preferably by two attachments. One attachment ensures the attachment of the retractor onto the support by cooperation of a support attachment part 27a-27b (FIG. 5B) and of a retractor attachment part (27a′-27b′).

In a preferred embodiment, the retractor is articulated on the support 12, by any means.

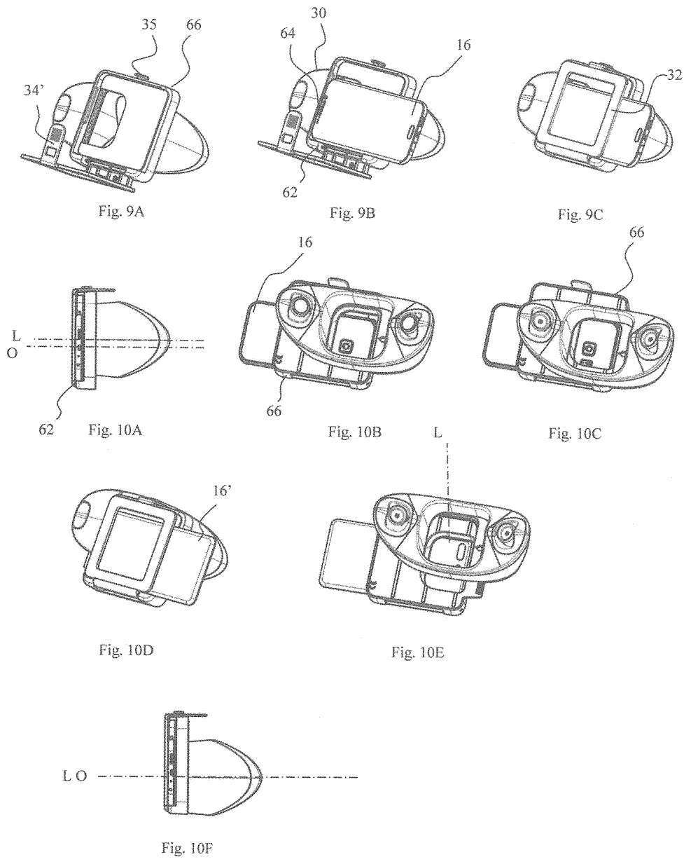

Preferably, the retractor 14 is mounted to rotate on the support. Preferably, as represented, the support comprises two support attachment parts 27a and 27b disposed symmetrically on either side of the median longitudinal plane P₁₂ of the support. Each support attachment part is mounted to pivot about a respective pivoting axis Ya and Yb (FIG. 11C), preferably exclusively about a respective pivoting axis Ya and Yb, preferably substantially horizontal, preferably forming an angle greater than 60° and less than 80° with the general plane of the retractor open 24. To this end, pivots 23a and 23b, of axes Ya and Yb, are secured to the housing, and the attachment parts are mounted to pivot about these pivots pivots 23a and 23b.

The pivots are said to be “secured to the housing” when they are rigidly fixed to the housing or fixed in such a way as to allow a guided displacement with respect to the housing, for example by pivoting about an axis, or by rotation, or by translation.

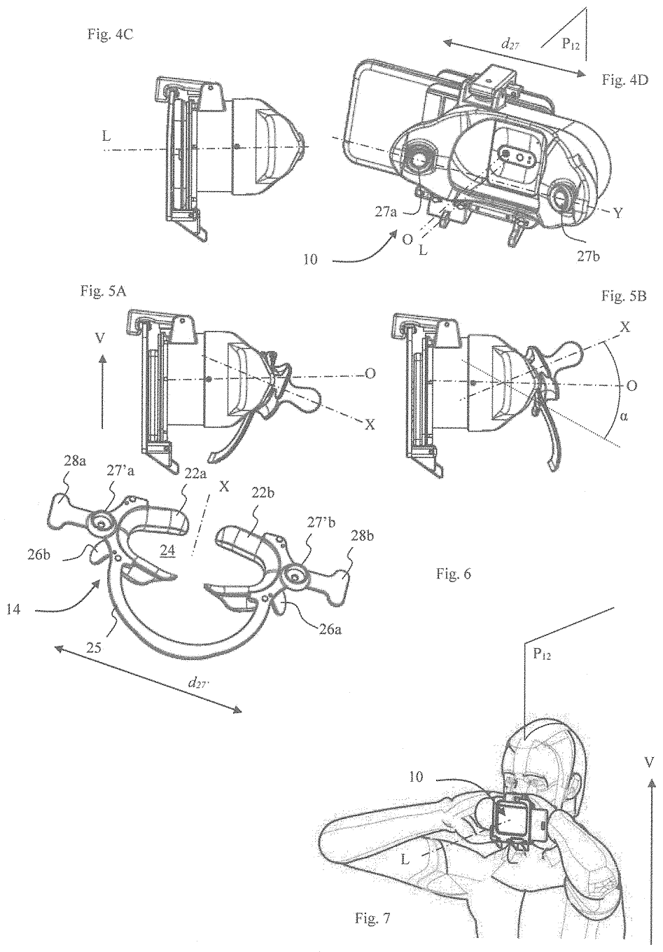

These pivotings allow a pivoting of the retractor about an axis Y (FIG. 4D), which considerably facilitates the taking of photos of the palate and of the tongue, by guiding the movements of the operator when taking photos.

Preferably, the maximum pivoting amplitude, represented by the angle α in FIG. 5B, is greater than 5°, even greater than 10°, and/or less than 30°, preferably less than 20° or 15°.

Preferably, the support comprises abutments configured to limit the amplitude of the pivoting. Preferably, the abutments are configured so that the extreme pivoting positions allow observation of all the teeth in the mouth of the patient.

The retractor is removable, that is to say that it can be mounted on and unmounted from the support by the operator. Advantageously, the same support can therefore be used for several retractors, and in particular for several retractors of different sizes.

Preferably, each support attachment part is magnetically attached to a corresponding retractor attachment part.

In one embodiment, a magnet of the retractor is disposed on either side of the median plane of the retractor and cooperates with a respect metal insert of the support, which ensures that position is maintained effectively. Preferably however, the configuration is reversed, the magnet being on the support and the metal inserts being in the retractor. Advantageously, the metal inserts of the retractor are therefore not attracted by the metal objects when the retractor is not attached to the support.

In one embodiment, the metal inserts are replaced by magnets oriented so as to increase the magnetic attraction.

Advantageously, the operator simply has to bring the support closer to the retractor for them to be secured to one another. The use of a magnetic attachment thus allows for a simple, reliable and very accurate attachment. Also, particularly advantageously, the use of a magnetic attachment makes it possible to attach the retractor to the support “blind”, which makes this attachment possible with no particular training.

More preferably, the attachments of the support to the retractor are conformed to ensure an attachment in a predetermined position of the support with respect to the retractor. In particular, one attachment part can have a conical form which, during the attachment operation, enters into a corresponding recess of the attachment part in order to ensure its centering with respect to said recess.

Preferably, the attachment parts of the retractor and of the support are conformed so that the retractor can be attached to the support only in a single predetermined position. The risk of error in assembling the retractor and the support is therefore reduced.

In one embodiment, as represented in FIGS. 11A, 11B and 11C, one attachment, preferably each attachment, comprises a retractor attachment part 27a′ and 27b′ that is movable in translation with respect to the support, along a translation axis Ta and Tb, respectively.

In the embodiment of FIG. 11C, each support attachment part 27a and 27b comprises a sleeve 29a and 29b, respectively, mounted to move in translation, along the translation axis Ta and Tb, respectively, on the body of the support.

In FIGS. 11A and 11C, the retractor attachment parts 27a′ and 27b′ are in position of maximum convergence with the support. In FIG. 11B, they are in the position of maximum separation from the support. The amplitude of the translation of the retractor attachment part with respect to the support attachment part is preferably greater than 0.5 cm and less than 2 cm, preferably less than 1 cm.

The translation axes Ta and Tb are preferably symmetrical with respect to the median longitudinal plane P₁₂ of the support. Preferably, each translation axis Ta and Tb forms, with the medial longitudinal plane P₁₂ of the support, an angle of less than 30°, preferably less than 20°, and/or preferably greater than 5°, preferably greater than 10°. Preferably, a plane at right angles to the axis Ta or Tb forms, with a plane at right angles to axis X of the retractor opening, an angle of less than 30°, preferably less than 20°, and/or preferably greater than 5°, preferably greater than 10°.

In the operating position, each retractor attachment part can therefore be separated from the support by pulling the retractor forward.

More preferably, the attachment comprises a return member for returning the attachment part of the support to the attachment part of the retractor, for example an elastic return spring or a magnet, preferably a magnet. In the absence of action by the operator, the two attachment parts are preferably pressed against one another.

Preferably, the retractor attachment part is movable by rotation and translation with respect to the support attachment part. Preferably, the orientation of the translation axis Ta—Tb is not modified by said rotation.

Preferably, the means for attaching the retractor to the support are configured so that, in the operating position, the retractor is elastically flexed about an axis V, preferably substantially vertical. The ergonomics are thereby enhanced.

Preferably, in the operating position, the angle formed by the two planes in which the two parts of the retractor (22a, 22b), respectively, extend, is greater than 120°, preferably greater than 140° and/or less than 170°, preferably less than 160°.

In one embodiment, the retractor is configured so that the angle formed by said two planes in which the two parts of the retractor (22a, 22b), respectively, extend, is greater than 120°, preferably greater than 140°, and/or less than 170°, preferably less than 160°, when said retractor is at rest, that is to say before being positioned in the operating position.

Preferably (FIGS. 4D and 6), the distance d_{27′} between the two retractor attachment parts is less the distance d₂₇ between the corresponding two support attachment parts, such that the retractor, when it is attached to the bearings of the support, is flexed. Preferably, the distance between the two retractor attachment parts is less by more than 2%, more than 5%, than the distance between the two support attachment parts.

### Cellphone

The cellphone 16 is a personal appliance, conventionally of substantially parallelepipedal form, conventionally comprising a camera.

The camera of the cellphone preferably provides color photos and/or infrared photos. The infrared photos advantageously make it possible to show the teeth with an excellent contrast.

In a preferred embodiment, the cellphone comprises an application allowing the operator to be guided in the operations, for example for indicating to him or her how to attach the retractor or the cellphone, or the orientation to be given to the support to take particular photos.

Support

The support 12 of the kit represented in FIGS. 1A-1B, 2A-2B, 3A-3B and 4A-4D comprises a housing 30, a door 32, or “cover” and a locking hook 34.

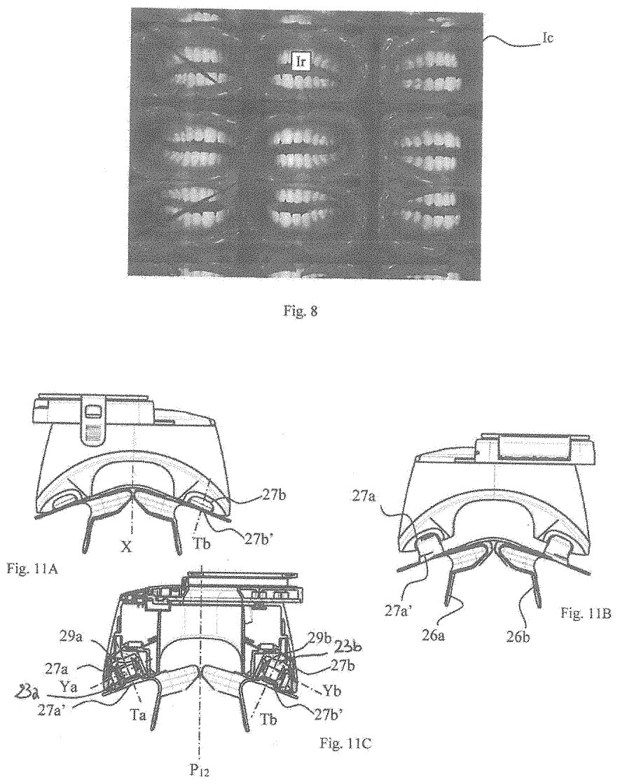

Preferably, the support bears a fixed mirror, preferably inside the housing 30, and oriented so that, in the operating position, the camera of the cellphone receives a composed image comprising a direct image of the retractor opening and an image of the retractor opening reflected by the mirror (FIG. 8). It thus simultaneously acquires several images of the teeth, observed from different angles. The photo acquisition procedure is thereby speeded up. More preferably, the support comprises, preferably inside the housing 30,
a colorimetric pattern and/or a translucency pattern, preferably a colorimetric pattern and a translucency pattern; and
preferably a light source oriented so as to illuminate, on the one hand, the teeth of the patient through the retractor opening and, on the other hand, said colorimetric pattern and/or said translucency pattern,

the support being configured so that, in the operating position, the camera receives an image of the retractor opening, on the one hand, and of said colorimetric pattern and/or of said translucency pattern on the other hand. Advantageously, the acquired photos thus make it possible to accurately determine the color and the translucency of the teeth and/or of the soft tissue such as the gums. In particular, the lighting can be controlled so as to limit disturbances by the light environment.

In the embodiment represented, the housing, of longitudinal axis L (FIG. 4C), emerges outward only on two opposing end faces, through a proximal opening 36, on the side of the cellphone, and a distal opening 38, on the side of the retractor, respectively (FIG. 1A, 1B, 3B). Preferably, the proximal and distal openings are substantially parallel. Preferably, in the operating position, they are substantially at right angles to the optical axis O of the camera.

Preferably, the length L₃₀ of the housing between the proximal and distal openings is less than 20 cm, 15 cm, 10 cm, 7 cm or 5 cm, and/or greater than 2 cm. A low length advantageously makes it possible to acquire photos of regions of the inside of the mouth close to the retractor.

Around the proximal opening 36, the housing defines a receiving face 40, preferably flat, for receiving the cellphone 16 (FIG. 1A).

The door 32 is mounted to move in rotation, on the housing, about an axis P, between open and closed positions. The axis P is preferably substantially horizontal and, in the operating position, preferably substantially parallel to the axis Y of rotation of the support on the retractor.

The cellphone is removably attached to the support 12. The attachment can therefore be reversibly deactivated, that is to say that the operator can, at will, secure the cellphone on and release it from the support. Preferably, the attachment of the cellphone is ensured by compression against the receiving face 40, by means of the door 32 in its closed position (FIG. 4A, 4B).

The receiving face 40 is preferably coated with a foam enhancing the friction with the cellphone. Preferably, the foam is elastically compressible, which allows the cellphone to be gripped against the receiving face 40 without damaging it.

Preferably, the door is locked, in the closed position, by means of the locking hook 34. To this end, the locking hook is mounted to move in rotation, on the housing, about an axis C, between the locked and unlocked positions in which it opposes and does not oppose the opening of the door 32, respectively (FIG. 1A). The axis C is preferably substantially horizontal and preferably substantially parallel to the axis Y of rotation of the support on the retractor.

In one embodiment, the locking hook and the housing comprises a magnet 44 and a metal insert, respectively, ensuring that the locking hook is held in the locked position (FIG. 4A, 4B).

FIGS. 9A-9C show an embodiment in which the locking hook takes the form of a tongue 34′, preferably elastic, pierced with a hole, and which cooperates with a pin 35 fixed onto the housing. After having positioned the cellphone (FIG. 9B), the insertion of the pin into the hole of the tongue keeps the door in the closed position (FIG. 9C).

Preferably, the door 32 comprises a window 46 through which the operator can see the screen on the back of the cellphone. The window can be a cutout or be composed of a transparent material. A cutout advantageously allows the operator to interact with the touch screen of the cellphone.

The door 32 preferably comprises a substantially flat bearing plate 48 and a foot 49 protruding from the general plane of the bearing plate 48, bearing a “door” pivot 50 defining the axis of rotation P of the door 32 (FIG. 1A, 2A).

Preferably, at least the parts of the bearing plate 48 which extend against the screen of the cellphone are transparent. The operator thus has a better view concerning the photo that he or she is about to take. Preferably all of the bearing plate 48 is transparent.

The internal face 52 of the bearing plate 48, which faces the receiving face 40 in the closed position, bears on the cellphone in the closed position. Preferably, the internal face 52 bears elastic blocks 54, preferably made of an elastomer, which avoid direct contact of the internal face 52 with the cellphone, and therefore limit the risk of damaging the latter or the bearing plate 48 (FIG. 1A, 3B).

The door pivot 50 is preferably mounted to slide on a sliding axis Z at right angles to the axis of rotation P and, preferably at right angles to the general plane of the receiving face 40.

The door pivot 50 is preferably mounted to slide in a rail 56 formed on the housing or in the door, preferably in the foot (FIG. 3b), so that the distance d between the door pivot 50 and the general plane of the bearing plate can be modified when the bearing plate 48 extends parallel to the receiving face 40.

Likewise, the door can comprise a rail of axis Z in which a pin of the housing slides.

Advantageously, cellphones of different thicknesses can be attached to the support, without risk of damage. Preferably, a spring is disposed in the rail 56 so as to elastically push or pull the door pivot 50 toward the proximal opening of the housing. Advantageously, no specific manipulation is therefore needed for the position of the door pivot 50 to be adapted to the thickness of the cellphone. Furthermore, advantageously, the elastic return of the door pivot ensures, in the closed position, a compression of the cellphone between the bearing plate 48 and the receiving face 40.

In the open position, the door leaves the receiving face 40 accessible for the operator to place the cellphone thereon or remove it therefrom.

Preferably, in the open position, when the door rests flat on a flat substrate S, preferably via at least one spacer 58 attached to the bearing plate 48, the housing 30 bears, by at least one bearing zone 60, on said substrate. Preferably, the angle θ between the plane of the receiving face 40 and the plane of the bearing plate is greater than 110°, 120°, even 125° and/or preferably less than 150°, 140°, even 135°. It is thus particularly easy to place the cellphone on the receiving face 40 or to remove it therefrom.

Preferably, the housing and/or the door 32 define a bottom abutment 62, disposed so that, in the open position, when the door rests flat on the substrate S, the bottom abutment 62 prevents the cellphone from slipping downward.

Preferably, the housing and/or the door 32 also define a lateral abutment 64, preferably a single lateral abutment 64, disposed so as to limit the lateral slipping of the cellphone, that is to say to the left in the embodiment represented.

When the cellphone is in contact with the bottom and lateral abutments, the camera observes the interior of the housing, in particular the distal opening of the housing. The operator therefore has no difficulty in correctly placing the cellphone.

Preferably, the support does not comprise any other abutments. The support can thus be used for cellphones of varied lengths and/or widths.

In one embodiment, as represented in FIGS. 9A-9C and 10A-10F, the cellphone can be displaced with respect to the housing.

Preferably, it is attached to a frame 66 which defines a bottom abutment 62 and a lateral abutment 64 ensuring a predetermined positioning of the cellphone when it is in contact with said abutments.

The optical axis O of the cellphone can be offset from the longitudinal axis L of the support, as represented in FIG. 10A. The mobility of the frame 66 advantageously allows these two axes to be aligned.

In one embodiment, the frame 66 can be moved only vertically, as represented in FIGS. 10B and 10C. Preferably, the frame comprises a slider which is mounted to slide in a rail of the housing.

In one embodiment, the frame 66 can be moved only vertically and horizontally, which allows the cellphone to be positioned in any position in the plane of the frame.

When another cellphone 16′ is used (FIG. 10D-10F), the position of the frame can advantageously be adjusted to merge the optical axis O and the longitudinal axis L.

For this adjustment to be rapid, the frame preferably comprises identification marks and the housing preferably comprises a marker. An alignment of an identification mark on the marker advantageously makes it possible to accurately know the position of the frame with respect to the housing. Obviously, likewise, the identification marks can be borne by the housing 30 and the marker can be borne by the frame 66.

Preferably, the identification marks are determined in such a way that the alignment of a mark with a marker indicates an alignment of the optical axis of any cellphone of a particular model with the longitudinal axis of the housing. In other words, each cellphone model has a corresponding mark disposed in such a way that it is facing a marker and a telephone of this model attached to the frame is correctly positioned on the support.

### Operation

The operation of the kit devolves directly from the preceding description.

Initially, the operator opens the door 32 of the support. He or she can, for greater stability, place the support on a substrate, for example a table (FIGS. 1A and 1B).

The operator places the cellphone on the receiving face 40, so that it is in abutment on the lateral abutment 64 and on the bottom abutment 62 (FIGS. 2A and 2B).

The cellphone then blocks the proximal opening and the optical axis O of its camera passes through the distal opening, which allows it to take photos through the distal opening. After attaching the retractor, the cellphone can thus take photos through the retractor opening.

Then, the operator closes the door 32.

After the door 32 has entered into contact with the cellphone, the continuation of the closure thereof pulls on the door pivot 50 against the spring housed in the rail 52. When the door reaches the closed position, the foam 42 and the blocks 54 are elastically compressed. They encase, and therefore lock in position, the cellphone (FIGS. 3A and 3B). The door can also be elastically deformed (FIG. 4C) and thus contribute to the clamping of the cellphone. However, preferably the traction on the door pivot 50 is adapted so that, in the closed position, the door remains substantially flat.

Next, the operator folds back the locking hook to immobilize the door in closed position (FIGS. 4A, 4B, 4C and 4D). The locking hook is held in the locked position by the hooking of the housing and/or action of the magnet 44.

The operator sees the touch screen through the window 46 and, preferably, has access to the touch screen of the cellphone, which allows him or her, notably, to control the camera.

The operator also attaches the retractor to the support. This operation can take place before or after the attachment of the cellphone.

To this end, the operator brings the retractor close to the support to have the retractor attachment parts facing the corresponding support attachment parts.

The magnetic attraction activates the attachment rapidly, accurately and with no particular precaution. Upon this activation, the guidance and/or the form of the parts of the attachment ensure a predetermined positioning of the retractor with respect to the support.

The retractor then at least partially encircles the center of the distal opening, such that the retractor opening and the distal opening are at least partly superposed.

The kit is then ready to be used (FIGS. 5A and 5B).

In this position, the support can be moved in rotation around the retractor, about the axis Y.

The patient, who can also be the operator, then arranges his or her lips in the gutters defined by the rim 22 of the retractor (FIG. 7). The retractor is conventionally configured so as to elastically separate the upper and lower lips of the patient from one another so as to free the teeth and make them visible through the retractor opening. In particular, unlike a jaw retractor, the retractor does not bear on the teeth so as to separate the two jaws from one another. The retractor therefore allows the lips to be pulled up. The retractor thus allows the teeth to be observed without being hampered by the lips. The relative mobility of the two parts 22a and 22b of the rim and the elasticity of the handle 25 facilitates this operation.

The teeth of the patient are then clearly freed, notably by virtue of the lugs 26a and 26b. The closed housing advantageously masks the teeth, which preserves the intimacy of the patient. The camera of the cellphone sees an image, and even if the support comprises a mirror, a composed image I_{c} comprising a direct image I_{d} and one or more reflected images Iᵣ through the mirror or mirrors (FIG. 8).

The retractor does not prevent the jaws from being closed together or moved apart.

It thus allows the acquisition of photos, representing the teeth of the patient, while the patient has the mouth open or the mouth closed, by retaining the separation of the lips.

By pressing on the trigger of the acquisition device, the operator acquires one or more photos.

By pivoting the support about the axis Y, the camera can observe different regions of the mouth. In particular, when the pivoting axis Y is horizontal, it allows photos to be taken of the dental arch in determined conditions.

Preferably, the segment of the axis Y which extends between the two attachments of the support (27a, 27b), extends between the two horizontal planes which pass through the bottom and top ends of the retractor opening respectively.

Preferably, it extends to less than 5 cm, 2 cm, 1 cm from the horizontal plane which passes through the optical axis O and/or which passes through the longitudinal axis L.

Preferably, said segment is separated by less than 10 cm, 5 cm, 3 cm, 2 cm from the distal opening.

Preferably, the pivoting axis of the support with respect to the retractor is substantially within a plane at right angles to the longitudinal axis L and/or to the optical axis O, and preferably horizontal. Notably, the distance between the camera and the retractor opening is preferably substantially constant.

Preferably, the distance between the camera and the retractor opening is less than 20 cm, 15 cm, 10 cm, 7 cm or 5 cm, and/or greater than 2 cm.

A low distance advantageously allows photos to be acquired of regions of the inside of the mouth close to the retractor.

That advantageously makes it possible to augment the accuracy of the taking of the photos during the procedure.

Furthermore, the setting of the camera and the subsequent analysis of the acquired photos are thereby facilitated.

The possibility of making a retractor attachment part translate along the axis Ta or Tb makes it possible to take lateral photos comfortably, without excessive deformation of the mouth.

In one embodiment, the taking of several successive photos results from a single actuation of the trigger of the camera. In particular, the camera can be configured to successively take several photos, with different focal distances, as a consequence of a single trigger.

Moreover, if necessary, the camera acquires a photo of the teeth of the patient also representing the colorimetric and translucency patterns, which allows for a correction of the colors of the photo. For the correction to be accurate, the light source is preferably set so that the patterns are illuminated in the same conditions as when measuring real properties. The use of a closed housing facilitates the control of the lighting.

As now clearly emerges, the invention considerably facilitates the acquisition of photos, including photos representing the vestibular faces of molars, in particular for the implementation of the method described in PCT/EP2015/074896. It allows for a very rapid acquisition of several photos, typically in less than a minute, without requiring a specialist person, notably a dentist or an orthodontist. The acquisition of photos can in particular be performed by the patient him or herself or by someone close to him or her, with a simple cellphone, anywhere, and in particular outside of a medical, dental or orthodontic practice. Furthermore, the acquisition is possible without the use of a tool bearing on the ground to immobilize the cellphone, and notably without tripod.

Furthermore, it allows for a rapid and reliable attachment of the cellphone, regardless of its thickness, without risk of damaging it.

Finally, the guided mobility of the retractor with respect to the cellphone makes it possible to easily take photos of regions of the mouth that are difficult to access.

Obviously, the invention is not limited to the embodiments described and represented, which are for purely illustrative purposes.

In particular, the form of the housing is not limiting.

The articulation is not limited to a pivot link. In one particular embodiment, the support is mounted with ball joint on the retractor.

Preferably, the center of the rotation of the support with respect to the retractor lies within a horizontal plane which extends between two horizontal planes which pass through the top and bottom ends of the retractor opening respectively. Preferably, the center of the rotation of the support with respect to the retractor lies within a plane which extends less than 5 cm, 2 cm, 1 cm from the horizontal plane which passes through the optical axis O, and/or which passes through the longitudinal axis L.

Preferably, the center of the rotation of the support with respect to the retractor is situated on the optical axis and/or on the longitudinal axis.

That allows for a better accuracy in the taking of the photos during the procedure.

The support can be attached to the retractor by any means, for example by means of clips, and/or self-gripping strips of Velcro® type, and/or clamping jaws, and/or screws and/or magnets, and/or by complementarity of form between the support and the retractor.

The cellphone can be attached to the support by any means, for example chosen from the group composed of snap-fitting means, self-gripping strips of Velcro® type, clamping jaws, screws, magnets, and complementarity of form between the support and the cellphone.

The camera is not limited to the camera of a cellphone. Any other personal device commonly available on the market, comprising a camera can be used, for example a so-called “connected” camera, a so-called “smart” watch, or a tablet or a personal computer, fixed or portable, comprising an image acquisition system, such as a webcam or a camera, preferably a digital camera. It preferably weighs less than 3 kg, less than 2 kg, less than 1 kg, less than 500 g, preferably less than 300 g.

1. A dental photography kit comprising:
a support, taking the form of a housing, comprising a support attachment part;
a dental retractor defining a retractor opening and comprising a retractor attachment part that is removably attached to the support attachment part when the retractor is in the mounted position on the support; and
a camera removably attached to the support in an operating position in which the camera is oriented to receive an at least partial image of the retractor opening,
characterized in that the retractor attachment part is articulated on the support attachment part.

2. The kit as claimed in claim 1, wherein the retractor attachment part is mounted to pivot on the support attachment part.

3. The kit as claimed in claim 2, wherein the retractor attachment part is mounted to pivot on the support attachment part about a pivoting axis which, in the operating position, extends horizontally.

4. The kit as claimed in claim 2, wherein the pivoting axis is translationally movable with respect to the support in a direction of translation that is different from that of the pivoting axis.

5. The kit as claimed in claim 2, wherein the maximum amplitude of pivoting of the support with respect to the retractor is greater than 5° and less than 30°.

6. The kit as claimed in claim 1, wherein the retractor attachment part is magnetically attached to the support attachment part.

7. The kit as claimed in claim 6, wherein the retractor bears two retractor attachment parts that are removably attached to two support attachment parts, respectively, the distance between the two retractor attachment parts being less than the distance between the two support attachment parts, such that the retractor is flexed.

8. The kit as claimed in claim 6, wherein the retractor attachment parts and support attachment parts are conformed in such a way that the retractor can be attached to the support only in a single predetermined position.

9. The kit as claimed in claim 6, wherein the retractor attachment part comprises a metal insert and the support attachment part comprises a magnet disposed to attract the metal insert.

10. The kit as claimed in claim 1, wherein the support comprises
a body, preferably in the form of a housing, and
a door mounted to pivot on the body about a door pivot of axis, between a closed position in which it holds a cellphone clamped against a receiving face of the body, and an open position in which it allows the cellphone to be extracted,
the door pivot being mounted to slide in translation, along a sliding axis not parallel to the axis, with respect to the body of the support.

11. The kit as claimed in claim 10, comprising a return member disposed so as to oppose, preferably elastically, a separation of the door pivot away from the plane of the receiving face.

12. The kit as claimed in claim 10, wherein, in the open position of the door, the cellphone rests on the receiving face of the support, and wherein the support comprises only first and second abutments,
the first abutment limiting the slipping of the cellphone on the receiving face in a first direction and in just one direction, when the door is in the open position, and
the second abutment limiting the slipping of the cellphone on the receiving face, in a second direction, different from the first direction, and in just one direction, when the door is in the open position.

13. The kit as claimed in claim 12, wherein the housing emerges outward on only two opposing end faces, through a proximal opening, on the side of the cellphone, and a distal opening, on the side of the retractor.

14. The kit as claimed in claim 1, wherein the retractor comprises cheek separating lugs.

15. The kit as claimed in any claim 1, wherein the distance between the camera and the retractor opening is substantially constant, preferably less than 20 cm, 15 cm, 10 cm, 7 cm or 5 cm and/or greater than 2 cm.
